(19) United States
(12) Patent Application Publication
(10) Pub. No.: US 2012/0137398 A1
(43) Pub. Date: Jun. 7, 2012

(54) OUTER LENS FOR GOGGLES

(75) Inventor: Derek Leslie Arnold, Emmeloord (NL)

(73) Assignee: HD INSPIRATION HOLDING B.V., LELYSTAD (NL)

(21) Appl. No.: 13/321,851

(22) PCT Filed: May 21, 2010
(86) PCT No.: PCT/EP2010/057081

§ 371 Date: Feb. 22, 2012

(30) Foreign Application Priority Data
May 22, 2009 (GB) 0908893.1

(51) Int. Cl. A61F 9/02; A42B 3/04

(52) U.S. Cl. 2/10; 2/431; 2/435; 2/432; 2/12

(57) ABSTRACT

There is provided a goggle assembly (1) comprising: a frame; an inner lens retained by the frame (5); an outer lens (3) having a first relaxed configuration and a second pre-tensioned configuration; and attachment elements (7) releasably retaining the outer lens in its second configuration in spaced relation in front of the inner lens.

### BACKGROUND OF THE INVENTION

1. Field of the Invention

The present invention relates generally to eyewear and more particularly to goggles, especially those worn for skiing and other winter sports. The invention relates further to a lens element that can be detachably fitted at an outer side of such eyewear.

2. Description of the Related Art

Eyeglasses and goggles of many forms are well known in the art. In general, such items are used for protection from foreign objects or radiation and/or for correcting or improving vision. Increasingly, these items additionally have a fashion purpose and may be used to enhance the user's style or image. The term eyewear has recently been used as a general purpose term encompassing all types of glasses (also known as spectacles), shaded glasses and goggles.

Goggles are generally understood to be a form of eyewear that attach around the back of the head using a strap, which is usually elasticated. A lens or lenses in front of the eyes are spaced from the face by a frame. The frame may cover the nose (sometimes referred to as a mask) but more generally it sits above the nose. The frame usually has a flexible edge in order to better conform to the shape of a user's face. Some goggles are applied over helmets, for example ski helmets or motocross helmets, in which case the strap is pulled around the back of the helmet.

One major area where goggles are extensively used is in the field of winter sports. Ski goggles are used to protect from the sun's glare, to reduce the harmful effects of UV-radiation and to protect the eyes from cold and precipitation. A good pair of goggles may be considered essential in order to be able to safely and comfortably participate in winter sports in weather conditions that may be very changeable. Such goggles should be comfortable to wear and easy to use and should be resilient against breakage by being dropped, crushed or scratched. Of particular significance, it is highly desirable that goggles do not fog or mist up in use. Such fogging may occur due to relatively high humidity levels of the air within the goggles adjacent to the user's face, often resulting from exertion of the wearer leading to sweating. In very cold conditions, condensation onto the inner surface of the relatively cold lens leads to fogging. Some goggles have sought to overcome this problem by the use of anti-fog coatings. Coatings alone are however generally less than adequate and are easily damaged (especially by lens cleaning) whereby the vision through the lens is disrupted and the goggles must be discarded. In particular it is common for users to wipe away condensation on the inside of the goggles, which action results in scratching or removal of the thin anti-fog coating. Once the anti-fog coating is scratched or removed the goggles readily fog up.

A number of products are available that provide improved fog resistance by the use of a double lens assembly. One such arrangement is shown in U.S. Pat. No. 3,718,937 in which an inner lens is retained by a groove formed in a flange part of the outer lens. The inner lens may be omitted if desired. Another assembly is shown in U.S. Pat. No. 7,404,217. According to that disclosure, inner and outer lenses are permanently bonded together by a rubber seal. The sealed unit is held in the goggle frame by engagement of the edge of the outer lens in a peripheral groove in the frame.

In certain situations it may be desirable to remove a lens in order to replace it with a different one. This may be the case if the lens has become broken but may also be in order to provide a different functionality or style. In particular, a lens may be replaced with one of increased light resistance or by one having a different colour, for example to offer different visual effects as might be needed in various light conditions; or in order to match a clothing style or mood. One system that allows a lens assembly to be replaced is shown in US2007/0153230. The system is relatively complex and cannot easily be fitted to existing goggle designs. It also requires replacement of the complete lens assembly. Another system is shown in U.S. Pat. No. 3,012,248 in which an outer lens structure may be fitted to the outer surface of an existing goggle lens. The outer lens structure comprises a sealing edge in the form of an elastomeric gasket and is held against the inner lens by the straps. Although this arrangement is relatively simple, sealing is dependent upon the strap tension and removal of the outer lens requires separation from the straps, and the sealing edge enters the field of vision of the wearer. The two lenses are also sealed with an airtight chamber between.

A protective goggle arrangement is known from U.S. Pat. No. 5,802,622 having a pair of protective shields for ballistic and radiation protection. The outer radiation shield is removably connected to the goggle frame by prongs at its ends. The outer shield forms a seal against the frame. Although this design appears to fulfill the requirements of easy exchange of the outer lens, it requires a dedicated frame and cannot be retrofitted to existing designs. It also relies on the outer surface of the frame for sealing, which may not always be conveniently smooth for this purpose. Bending of the frame in use may also lead to opening of the seal.

Thus, there is a particular need for a goggle assembly in which an outer lens may be provided in a simple and convenient manner.

### BRIEF SUMMARY OF THE INVENTION

In one form, the present invention addresses these problems by providing a goggle assembly comprising: a frame; an inner lens retained by the frame; an outer lens having a first relaxed configuration and a second pre-tensioned configuration; and attachment elements releasably retaining the outer lens in its second configuration in spaced relation in front of the inner lens. By providing the outer lens with an initial pretension, a firm attachment may be achieved as the outer lens exerts a constant restoring force over the front surface of the frame or inner lens. In the present context, reference to the term “goggle” is preferably intended to include any eyewear comprising the features as defined in the claims and is not intended to be limiting to devices held against the face by elastic straps. In a preferred application of the invention, the term “goggle” refers to the type of eyewear in which a lens or lenses are located in front of the eyes and are spaced from the face by a frame that at least partly forms a closed space about the eyes and possibly also about a portion or more of the face. The frame may cover the nose (sometimes referred to as a mask) but more generally it sits above the nose. The frame usually has a flexible edge in order to better conform to the shape of a user's face.

In one embodiment, the assembly may comprise a sealing element located between the outer lens and the inner lens, forming a substantially sealed space there between so as to form a heat insulating barrier between the outer and inner lenses. The seal is preferably sufficient to form a fully airtight seal. The pretension in the outer lens may serve to press the sealing element into sealing relation with the inner lens or its frame over much of its length. Were no pretension present, the sealing effect would be unequal and leakage of air around the seal could result. Preferably the seal is located at the outer peripheral edge of the outer lens. In this manner, the unimpeded field of vision may be maximized. It is however also contemplated that it may be set inwardly from the peripheral edge. The edge region outside of the seal may then be used for attachment purposes or may extend in front of and cover the goggles frame.

According to one embodiment, the sealing element comprises a silicone bead attached to an inner surface of the outer lens. Such dry, set silicone material has been found useful and versatile in providing a releasable sealing engagement between lens elements. It may be accurately and efficiently applied using computer numerically controlled (CNC) machinery and subsequently dried and cured. Other methods of application are also considered.

Alternatively, the sealing element comprises an elastomeric edge, which may be integrally moulded with the outer lens. In this context, integrally moulded is intended to comprise any form of moulding procedure including over-moulding, dual injection moulding and the like. The skilled person will be well aware of the advantageous alternative seal shapes that may be achieved in such manner and the various materials that may be employed according to the precise properties required.

According to a further aspect of the invention, the attachment elements may be provided on the inner lens. Such an arrangement is particularly important in allowing retrofitting of a lens assembly of the present invention to an existing model of goggles. Since the attachment takes place between the inner and outer lenses, no change in design to the frame may be necessary. This particular feature is believed to be of itself novel and inventive in the context of dual lens goggles.

One particular form of attachment element that may be used in this manner is an insert pin or plug. The pin or plug may be inserted through a hole formed in the inner lens from inside outwards. It may also be resilient in order to seal the hole against ingress of moisture. It may be further provided with engagement surfaces for retaining the outer lens, preferably in tension (i.e. the pins are pulled by the pre-stress). The engagement surfaces may comprise enlarged heads, ridges, grooves or the like that can be engaged in suitable manner, e.g. by a pre-tensioned outer lens. To retain the outer lens, two pins may be provided at the left and right side of the viewing area. Alternatively, four pins may be mounted at corners of the lens or a plurality of pins may be distributed around the periphery.

In an alternative embodiment, the attachment elements engage with or are provided on the frame. Such attachment elements may comprise posts or pins fitted to or integrally formed with the frame. Such pins or posts may engage with the outer lens as described above. Alternatively, the engagement elements may comprise interlocking peripheral edges formed respectively on the frame and on the outer lens. Such an arrangement may be achieved in the case that an integrally moulded edge is provided on the outer lens.

In a yet further alternative, the outer lens may be held in pre-tension against the frame or inner lens by engagement with a strap for retaining the goggles on a user's head or helmet.

According to one aspect of the invention, the inner lens has a thickness of between 0.6 mm and 1.5 mm, preferably between 0.8 mm and 1.2 mm and most preferably about 1.0 mm. Conventional lens material used for goggles is around 0.6 mm or less. This material is relatively flimsy and not immediately suitable for serving as an anchoring location for attachment elements or for withstanding forces applied by a pre-tensioned outer lens. By choosing an inner lens of a significantly higher gauge, increased stiffness and resilience may be achieved for a construction of the type presently claimed.

Furthermore it is preferable that the inner lens is hydrophilic, at least at its inner surface. Various materials are known as suitable candidates for reducing fogging. In particular, cellulose acetate and cellulose propionate are recommended anti-fog materials. Alternatively, a less hydrophilic material may be used in combination with a hydrophilic coating on its inner surface. The skilled person will be well aware of various alternative materials that may be used for this purpose including silicone based material applied by dip-coating or the like.

Preferably each lens has a thickness of at least 0.6 mm. More preferably at least one of the lenses has a thickness of at least 0.8 mm; even more preferably at least 1.0 mm. The lenses preferably have thicknesses of at most 6 mm, more preferably at most 4 mm, even more preferably at most 3 mm, and most preferably at most 1.5 mm. The thickness of the lenses depends at least partially on the intended purpose of the goggles. Some goggles may be used as ballistic goggles, in which case thicker lenses are needed to offer protection against collisions. Winter sport goggles may typically require less thick lenses.

Preferably the outer lens has a thickness of between 0.6 mm and 1.5 mm, preferably between 0.8 mm and 1.2 mm and most preferably about 1.0 mm. The inner lens preferably has a thickness of between 0.8 mm and 2.0 mm, preferably between 1.0 mm and 1.8 mm and most preferably about 1.2 mm. An appropriate balance of force between the outer lens and the inner lens/frame combination is desirable in order to form a stable structure. This is particularly important where pre-tension of the outer lens is required to maintain a good seal. In certain cases a seal or attachment element may be integrally formed with the outer lens to form an outer lens arrangement. In these cases it will be understood that the combined strength of the outer lens arrangement will be important in determining its sealing relation with the remainder of the goggles.

In an embodiment where the attachment elements may be provided on the inner lens, the inner lens preferably has a thickness of at least 1.0 mm, more preferably at least 1.15 mm, even more preferably at least 1.5 mm, and most preferably at least 1.8 mm.

According to an important aspect of the invention, the outer lens is substantially flat or concave in its first configuration and is convex in its second configuration. In this context, it is understood that the terms convex and concave are used as viewed from the outside in front of the goggles. The outer lens in its convex second configuration is shaped to allow it to wrap around a user's face. The lenses may both be either two-dimensional (2D) or three-dimensional (3D). The skilled person will understand that an outer lens having a 2D shape is easily bent between first and second configurations. Nevertheless, a flexible 3D outer lens may also have two configurations between which it can flip.

According to an important aspect of the invention, the outer lens may be provided with a functional characteristic. In this context, functional characteristic is intended to refer to a function other than that provided by clear glass or polycarbonate (impact and ambient protection). The functional characteristic may be selected from the group comprising: scratch resistant, anti-fogging, glare reducing, tinted, mirrored, polarizing, photochromatic and electrochromatic. In particular, the outer lens may be provided in a plurality of different tints or colours. The shading/colouring in the tinted, mirrored, polarizing, photochromatic or electrochromatic versions of the outer lens may be homogeneous across the outer lens surface, or may be graded thereover. For example, one or both of the upper and lower areas of the goggles may have increased shading/colouring than the horizontal middle line, this being in order to reduce glare from sun (above) and reflection from snow or water (below) while leaving a middle line less shaded for better vision.

The invention also relates to an outer lens arrangement for affixing to a front side of a pair of goggles, the arrangement comprising: a goggles shaped outer lens, having an outer surface and an inner surface and a cutaway area corresponding to a user's nose, the outer lens in its unstressed condition being curved in a first sense such that the inner surface is convex; a peripheral seal located at least partially on the inner surface for engagement with the front side of the goggles on flexing the outer lens to be curved in a sense opposite to the first sense such that the inner surface is concave.

According to another distinctive feature of the outer lens arrangement of the present invention there may be provided a lip for facilitating removal of the outer lens from the goggles. The lip may be an extension of the outer lens or may be an over-moulded or integrally moulded element, or an additional pull ring or tab fixed or adhered to the lens.

The invention still further relates to a method of fitting an outer lens to a goggles assembly provided with a frame having an inner lens retained at its edge regions by the frame. The method comprises forming a plurality of openings through the inner lens in the edge regions; inserting attachment elements into the openings such that the attachment elements protrude outwardly from the inner lens; and flexing the outer lens into engagement with the attachment elements such that the outer lens is held in pre-tension in spaced relation with respect to the inner lens. In this manner, an exchangeable outer lens may be effectively retrofitted to an existing design without requiring a change to the frame of the goggles.

In a further alternative embodiment of the invention there is provided a double lens goggles apparatus comprising an outer lens, a goggles frame and an inner lens carried by the goggles frame, the outer goggles lens being releasably held against the goggles frame or inner goggles lens by a strap (preferably an elasticated strap) for retaining the goggles on a user's head or helmet. In such an embodiment the outer lens is provided with connectors at its sides for attachment to the strap. The connectors may take the form of holes for threading a strap therethrough, poppers, studs, raised posts or lips, or other suitable mechanical connectors. Preferably the connectors are holes in the outer lens itself, or are connectors located on the side of the outer lens closest to the wearer's face. Advantageously this allows the whole forward facing portion of the visor assembly to be substantially the outer lens, with minimal additional components showing.

The inner lens is provided with a frame (preferably elastomeric) along its periphery. This frame preferably carries connectors for releasably fixing the inner and outer lenses in relation to one another. These connectors preferably take the form of retaining lips, but may also be pins (as discussed above) poppers, studs, raised posts, magnets or other suitable connectors. Advantageously, since the inner lens and outer lens are so held in relation to one another there is no need to provide a direct fastening (for example threading holes) for direct attachment of the goggle retaining strap to either the frame or the inner lens.

When in use the strap is pulled over the head or helmet of a user and so pulls the outer lens into tension with the frame or inner lens. The frame is preferably shaped so that when the outer lens is pulled into tension therewith, a seal is formed therebetween and a sealed chamber is created between the two lenses with the frame as its boundary. Advantageously, since the seal is formed with the frame of the inner lens, no additional sealing spacing member need be provided on the outer lens to space it from or seal it against the inner lens. The breadth of the viewing field can thereby be maintained.

The frame further carries on its inner side (the side closest to the wearer) a face interacting portion for resting against the face of the user. Many face interacting portions are known and commonly comprise shaped foams of varying stiffness and thickness to form a comfortable fit. The face interacting portion may be integrally formed with the frame, or may be subsequently attached or bonded thereto.

### BRIEF DESCRIPTION OF THE DRAWINGS

The features and advantages of the invention will be appreciated upon reference to the following drawings, in which:

FIG. 1 is a perspective view of a pair of goggles according to one embodiment of the invention;

FIG. 2a is a view of the goggles of FIG. 1;

FIG. 2b is an enlarged view of the circled part of FIG. 2;

FIG. 3 is an exploded perspective view of the goggles of FIG. 1;

FIG. 4 is a perspective view of a pin useful in an embodiment of the invention;

FIG. 5 is a side view of a part of the goggles of FIG. 1;

FIGS. 6a-6c are schematic illustrations of part cross-sections through components of a visor assembly;

FIGS. 7a-7b are schematic illustrations of part cross-sections through components of a visor assembly;

FIG. 8 is a schematic illustration of a cross-section through a part of a visor assembly;

FIG. 9 is a schematic illustration of a cross-section through a part of a visor assembly;

FIG. 10 is a perspective view of a pair of goggles according to one embodiment of the invention;

FIG. 11a is a view of the goggles of FIG. 10;

FIG. 11b is an enlarged view of the circled part of FIG. 11;

FIG. 12 is an exploded perspective view of the goggles of FIG. 10;

FIG. 13 is a perspective view of a pair of goggles according to one embodiment of the invention;

FIG. 14 is a perspective view of goggles of FIG. 13 in second configuration.

### DESCRIPTION OF ILLUSTRATIVE EMBODIMENTS

The following is a description of certain embodiments of the invention, given by way of example only and with reference to the drawings.

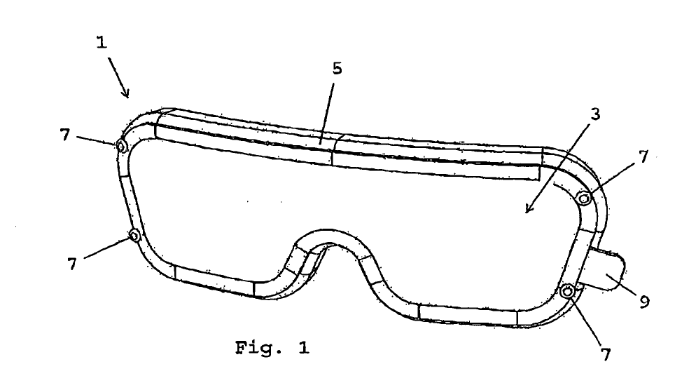

Shown in FIG. 1 is a pair of eye goggles 1 provided with an outer lens 3, a frame 5; and attachment elements in the form of pins 7 that releasably retain the outer lens on the frame 5. In use the frame 5 is brought into contact with a user's face about the eyes and is held there by e.g. an elastic strap (not shown) passed around the back of the head or a helmet.

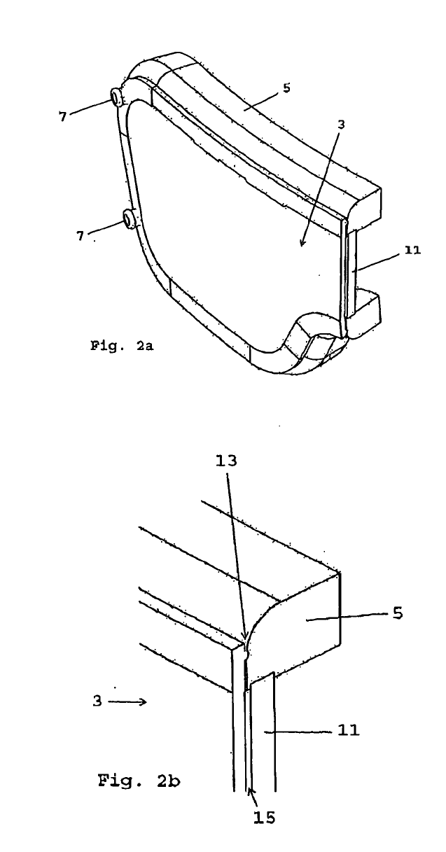

FIG. 2a shows a perspective cross-section through the goggles of FIG. 1, and FIG. 2b shows an enlarged view of the circled portion of FIG. 2b. As can be seen the goggles are provided with an inner lens 11 retained by the frame 5. The edge region of the outer lens 3 overlaps with the frame 5 and is provided with a seal 13 that extends along the periphery of the outer lens 3 and abuts the frame 5. The seal forms a substantially airtight barrier around the periphery of the outer lens 3 so that a sealed air-gap 15 is formed between the inner lens 11 and the outer lens 3. The air-gap 15 acts to insulate the inner lens 11 from the environment and thus helps to reduce fogging of the inner lens 11.

The seal 13 is bonded to the outer lens 3, but is either non-bonded or only temporarily bonded to the frame 5, to allow disengagement from the frame 5, so that the outer lens 3 can be removed.

In a less preferred embodiment (not shown) the seal 13 is replaced with a spacer that acts to space the outer lens 3 from the frame 5, but which does not form a sealed airspace. In such an embodiment the spacer does not extend continuously along the length of the periphery of the outer lens 3.

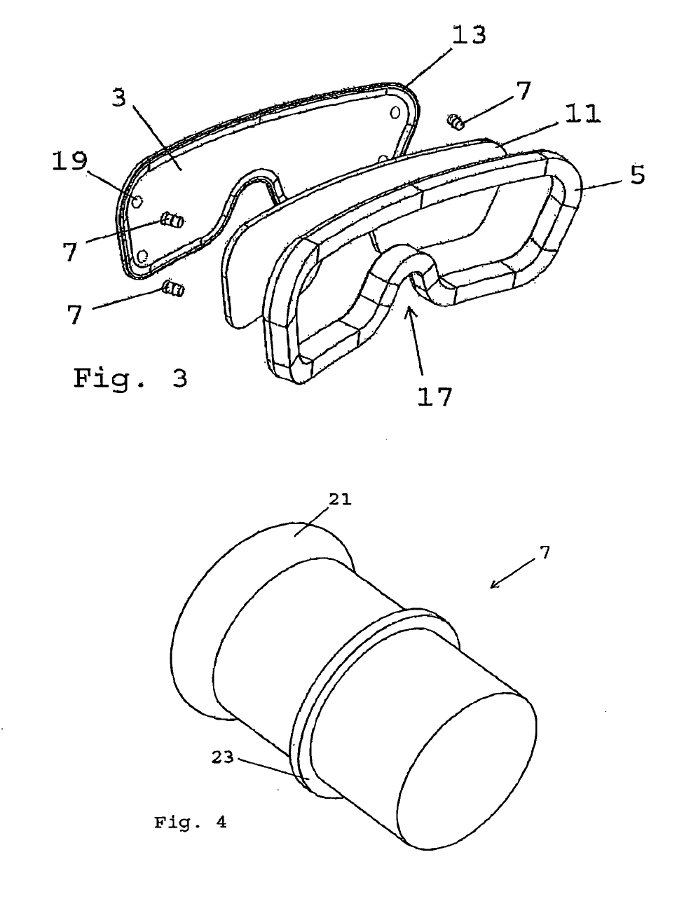

An exploded perspective of the goggles of FIG. 1 can be seen in FIG. 3.

The inner lens 11 is retained within the frame 5. The outer lens 3 is releasably retained by the pins 7. Four pins 7 (only three shown) are fitted into complementary sockets on the outer face of frame 5. The outer lens 3 is provided with four holes 19 (only three shown) corresponding to the pins 7 whereby the outer lens can be clipped onto the pins 7 and thus be anchored to the frame 5.

The outer lens 3 is pretensioned into concave form (as viewed from the outside in front of the goggles), so that when pulled into convex form (as shown in FIG. 3) and attached to the pins 7 it tends to pull the pins 7 in tension. This is advantageously believed to result in a good contact of the seal 13 with the frame 5

The pins are shown in enlarged detail in FIG. 4. Each pin 7 is provided with an distal ridge 21, and an proximal ridge 23. The distal ridge 21 forms a clip-over fastener for the holes in the outer lens 3, and the proximal ridge 23 forms a clip-over fastener for the sockets in the frame 5. In this manner the outer lens is releasably retained on the frame 5.

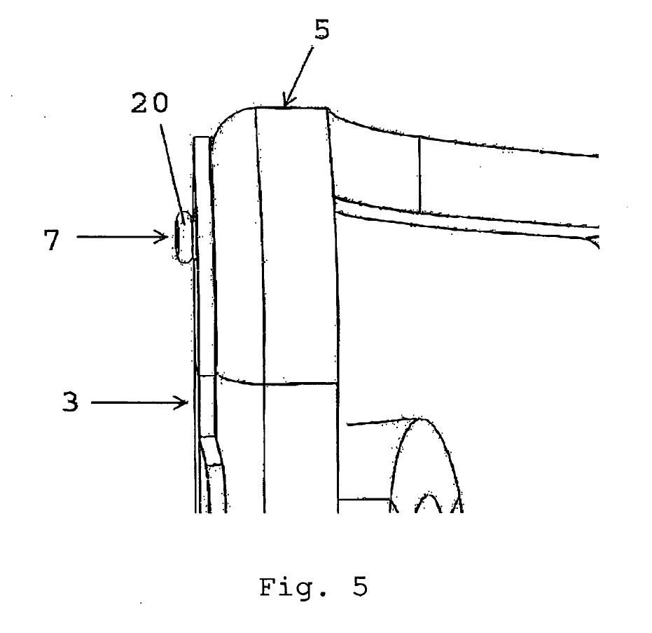

When anchored onto the frame 5 by pins 7, the outer lens 3 is snugly held against the frame 5 as shown in FIG. 5.

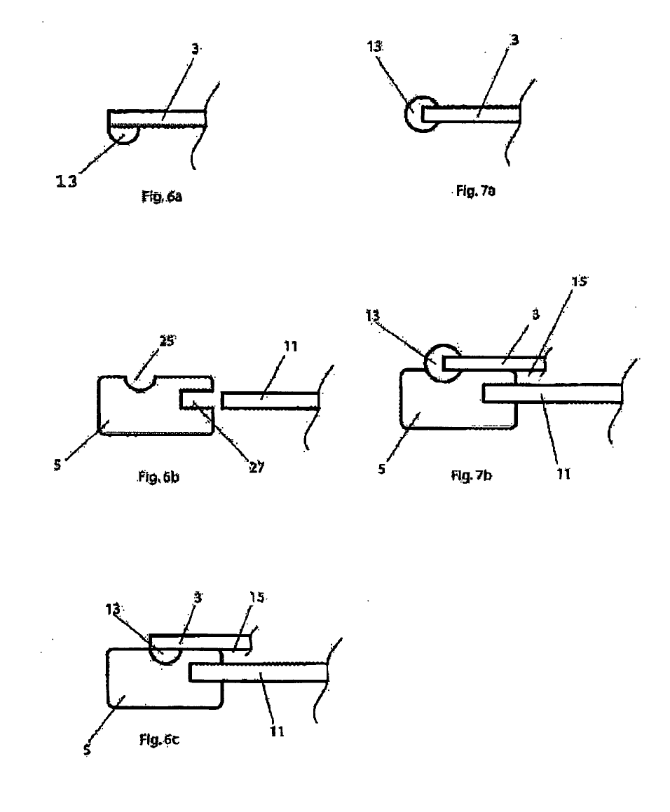

A variety of possible seal 13 configurations are illustrated in FIGS. 6 through 9.

In FIG. 6a a partial cross-section through the periphery of an outer lens 3 is shown. The outer lens 3 is provided with a seal 13 at the edge of its internal face. This outer lens 3 is combined with a frame assembly illustrated in FIGS. 6b and 6c. In FIG. 6b, an inner lens 11 is shown being fitted into a slot 27 in the frame 5. The frame 5 is further provided with a channel 25 complementary in shape and location to the seal 13. As seen in FIG. 6c the outer lens 3 is brought into abutment with the frame 5 and the seal 13 slots into the channel 25 thus forming an air-tight seal. Although some gap is shown in the channel 25 of FIG. 6c this is for illustrative purposes only, and in application, little or no gap is desired.

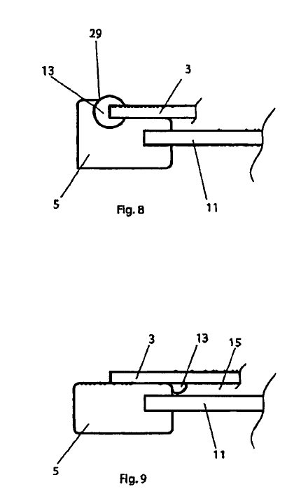

The embodiment of FIGS. 7a and 7b are similar to that of FIGS. 6a to 6c except that the seal 13 is applied to both the inner and outer surfaces of the outer lens 13. This allows the outer lens 3 to be reversible while still offering a sealed air-gap.

FIG. 8 shows an advantageous embodiment in which the outer lens 3 is releasably retained on the frame 5 by a clip in recess 29 complementary to a seal 13 or bead of material provided on the outer lens 3. In this embodiment pins 7 may or may not be provided, since the clip in recess 29 provides the attachment means.

The embodiment of FIG. 9 is provided with an outer lens 3 having an peripheral seal 13 spaced from its edge, and located to engage with a corner of the frame 5. Advantageously in such an embodiment, since the frame 5 does not require a channel complementary to the seal 13, such an outer lens 13 can be retro-fitted to pre-existing goggle frames.

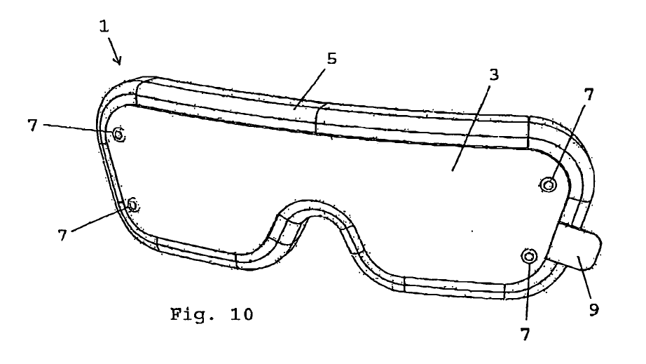

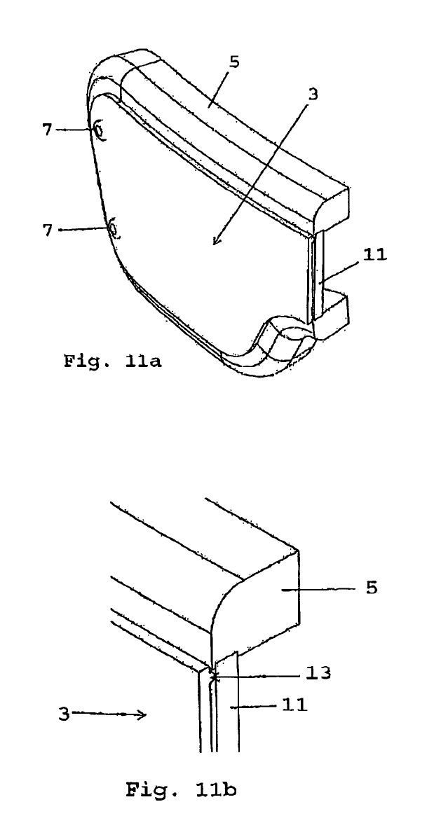

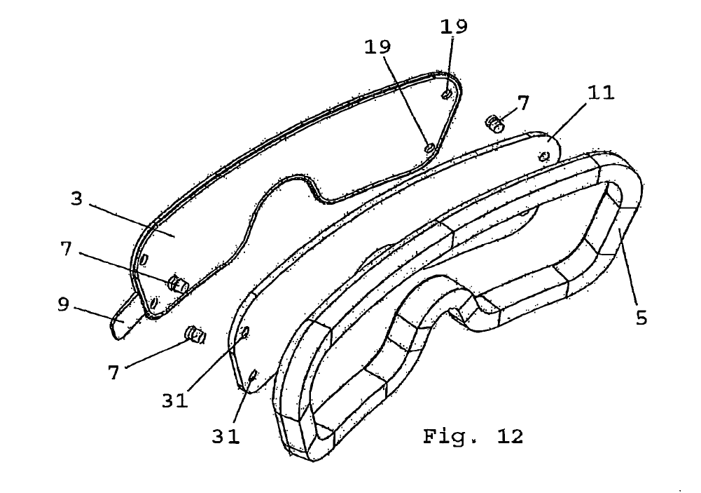

FIG. 10 shows a pair of goggles similar to that shown in FIG. 1 except that the outer lens 3 is releasably retained by attachment elements (pins 7) provided on the inner lens 11, and that the seal 13 releasably abuts the outer surface of the inner lens 13. This is most clearly seen in FIG. 11b, which is an enlarged view of the circled portion of FIG. 11a. Again as sealed air-gap 15 is formed between the outer lens 3 and inner lens 11. The seal 13 is bonded to the outer lens 3, but is either non-bonded or only temporarily bonded to the inner lens 11, to allow disengagement therefrom, so that the outer lens 3 can be removed.

The exploded view of claim 13 shows the components of this embodiment. The inner lens 11 is retained within the frame 5. The outer lens 3 is releasably retained by the pins 7. Four pins 7 are each fitted into holes 31 in the inner lens 11. The outer lens 3 is provided with four holes 19 corresponding to the pins 7 whereby the outer lens can be clipped onto the pins 7 and thus be anchored to the inner lens 11. Similarly to the embodiment of FIG. 3, the outer lens 3 is pretensioned into concave form.

The same pins 7 are used as illustrated in FIG. 4.

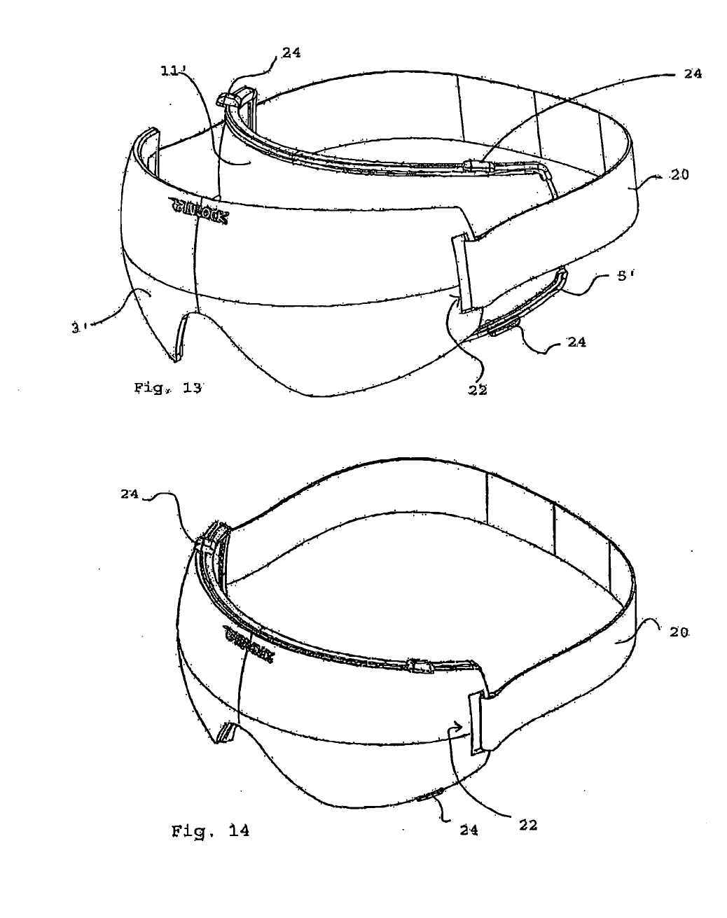

As can be seen in FIGS. 3 and 13 the illustrated embodiment has a mutually aligned cutaway portions in each of the outer lens, the inner lens and the frame. The cutaway portion 17 corresponds to a user's nose, and can be formed by a variety of procedures including direct formation of the arcuate recess in a mould or by stamping or cutting from a sheet of material.

Referring to FIGS. 1 and 10 the outer lens 3 is further provided with an integrally formed lip 9 to facilitate removal of the outer-lens 3.

FIG. 13 shows a goggles assembly provided with an outer lens 3′, a frame 5′, and an inner lens 11′. The inner lens is held in frame 5′ in a sealing manner to create a sealed chamber between the lenses. The frame 5′ may be separately formed and fitted to inner lens 11′ or may be integrally formed with inner lens 11′ by over-moulding or dual-injection moulding as discussed above.

The outer lens 3′ is provided with connection elements 22 for attachment to an elasticated strap 20 for passing around the back of a wearer's head or helmet. The strap 20 is preferably provided with an adjustable buckle (not shown) for varying its length to fit different helmet/head sizes. Such buckles are commonly known.

In the illustrated embodiment the connection elements 22 take the form of holes through which the strap 20 is threaded. As discussed above, other connection elements are envisaged and may be used.

In the illustrated embodiment the holes of the connection elements 20 create entry ports to a chamber between the two lenses. To seal this chamber, plugs are preferably provided to close the holes. Alternatively, the outer lens 3′ may be extended at its side edges such that the holes fall outside the periphery of the frame 5′. Since the seal of the outer lens 3′ is formed against the frame edge, locating the holes beyond this edge obviates the need for plugs.

The frame 5′ additionally carries fastening lips 24 for slotting onto the edges of the outer lens 3′, so as to at least loosely hold the outer lens 3′. In FIG. 14, the goggles assembly is shown with the outer lens 3′ held by the lips 24 of the frame 5′.

Further modifications in addition to those described above may be made to the structures and techniques described herein without departing from the spirit and scope of the invention. Accordingly, although specific embodiments have been described, these are examples only and are not limiting upon the scope of the invention.

1. A goggle assembly comprising:
a frame;
an inner lens retained by the frame;
an outer lens having a first relaxed configuration and a second pre-tensioned configuration; and
attachment elements releasably retaining the outer lens in its second configuration in spaced relation in front of the inner lens.

2. The assembly of claim 1, wherein the attachment elements are provided on the inner lens.

3. The assembly of claim 2, wherein the attachment elements comprise inserts protruding through holes formed through the inner lens.

4. The assembly of claim 1, wherein the attachment elements engage with the frame or with a strap for retaining the goggles on a user's head or helmet.

5. The assembly of claim 1, wherein the attachment elements comprise an edge integrally formed with the outer lens and a corresponding edge formed on the frame, which engage together.

6. The assembly of claim 1, further comprising a sealing element located between the outer lens and the inner lens and forming a substantially sealed space there between.

7. The assembly of claim 6, wherein the sealing element comprises a silicone bead attached to an inner surface of the outer lens.

8. The assembly of claim 6, wherein the sealing element comprises an elastomeric edge, integrally moulded with the outer lens.

9. The assembly of claim 1, wherein the inner lens has a thickness of between 0.6 mm and 1.5 mm.

10. The assembly of claim 1, wherein the inner lens is hydrophilic, at least at its inner surface.

11. The assembly of claim 1, wherein the outer lens has a thickness of between 0.6 mm and 1.5 mm.

12. The assembly of claim 1, wherein the outer lens is substantially flat or concave in its first configuration and is convex in its second configuration.

13. The assembly of claim 1, wherein the outer lens is provided with a functional characteristic selected from the group comprising: scratch resistant, anti-fogging, glare reducing, tinted, mirrored, polarizing, photochromatic and electrochromatic.

14. (canceled)

15. (canceled)

16. A method of fitting an outer lens to a pair of goggles comprising:
providing a frame having an inner lens retained at its edge regions by the frame;
providing a plurality of openings through the inner lens in the edge regions;
inserting attachment elements into the openings such that the attachment elements protrude outwardly from the inner lens;
flexing an outer lens into engagement with the attachment elements such that the outer lens is held in pre-tension in spaced relation with respect to the inner lens.

17. (canceled)

18. (canceled)

19. (canceled)

20. (canceled)

21. (canceled)

22. A kit of parts for constructing a goggle assembly, comprising:
a goggle frame;
an inner lens adapted to be retained by the goggle frame;
an outer lens having a first relaxed configuration and a second pre-tensioned configuration; and
attachment elements adapted for releasably retaining the outer lens in its second configuration in spaced relation in front of the inner lens.
